(19) United States
(12) Patent Application Publication
(10) Pub. No.: US 2021/0393814 A1
(43) Pub. Date: Dec. 23, 2021

(54) Wearable Sanitization Device

(71) Applicants: Allan Mark Klepfisz, Tel Aviv-Yafo (IL); Lance Ford, Pound Ridge, NY (US); Tomas Varga, Bratislava (SK)

(72) Inventors: Allan Mark Klepfisz, Tel Aviv-Yafo (IL); Lance Ford, Pound Ridge, NY (US); Tomas Varga, Bratislava (SK)

(21) Appl. No.: 17/355,012

(22) Filed: Jun. 22, 2021

Related U.S. Application Data
(63) Continuation-in-part of application No. 29/738,425, filed on Jun. 17, 2020.

(51) Int. Cl. A61L 2/00; A61L 2/26; A61L 2/24; A44C 5/00

(52) U.S. Cl. CPC A61L 2/0088; A61L 2/26; A61L 2/24; A61L 2202/121; A61L 2202/16; A61L 2202/15; A44C 5/0007

(57) ABSTRACT

A wearable sanitization device can be conveniently carried on one's body, especially the wrist. The preferred form of the apparatus involves a rubber or flexible plastic holster in the shape of a watch. A band connected to the holster may be adjusted to fit the shape and size of a user's wrist. A sanitizer-storing receptacle is mounted within the holster, thereby reducing the likelihood of misplacing sanitizer. A dispenser extending from the receptacle is oriented towards the hand of the user, ensuring the contents of the receptacle are dispensed appropriately upon the user's palm and fingers.

An exposed actuator, usually a button, on the receptacle may be depressed or actuated to enable easy, single-digit dispensing of the sanitizer. This also enables the apparatus to disperse hand sanitizer without having to be removed, thereby further preventing the apparatus from being misplaced and rendering two-hand handling of a sanitizer bottle obsolete.

The current application claims a priority to the U.S. Provisional Patent application Ser. No. 63/014,010 filed on Apr. 22, 2020. The U.S. provisional patent application 63/014,010 is revived within the two-month period for unintentional abandonment by Jun. 22, 2020.

The current application is also a continuation-in-part (CIP) application of the U.S. design application Ser. No. 29/738,425 filed on Jun. 17, 2020.

### FIELD OF THE INVENTION

The present invention relates generally to a device for dispersing sanitizer. More specifically, the present invention relates to a device that can be worn or carried for convenience in dispensing sanitizing fluid, especially upon the wearer's palms and fingers.

### BACKGROUND OF THE INVENTION

With the advent of ever-evolving dangerous viruses and bacteria, people increasingly feel a need to regularly sanitize their hands. They should do so reliably, regularly and frequently in order to minimize exposure to potentially-dangerous germs and bacteria. Among the better ways to prevent the spread of a pandemic is to practice washing one's hands. Good handwashing can kill off the pathogens responsible for pandemics and constantly doing it limits the spread of said pathogens. Unfortunately, there is not always an opportunity to practice good handwashing.

One of the most common substitutes for handwashing is using hand sanitizer. Hand sanitizer can be carried in small, portable containers that can be used when handwashing is not an option. By simply rubbing one's hands with hand sanitizer, a user can obtain many of the benefits of good handwashing without requiring a sink of running water. The obvious impediment to such regular sanitization is the need to have a bottle of sanitizer on hand. To facilitate regular sanitizing, manufacturers provide small bottles that easily fit into a pocket or purse. However, these are both easy to forget when leaving the house and inconvenient to access in one's pocket, often requiring a degree of fumbling to extract the sanitizer bottle. Furthermore, due to the size of the containers, the hand sanitizer bottles can often be misplaced. This can be problematic as one might not always have a supply of hand sanitizer available when one needs to use hand sanitizer. Therefore, there is a need for a device for dispersing hand sanitizer that can be conveniently carried on one's body.

The present invention addresses these issues. The present invention is a device for dispersing hand sanitizer that can be conveniently carried on one's body, especially the wrist. The preferred form of the present invention involves a rubber device in the shape of a watch. This device facilitates both providing readily available antiseptic or sanitizer as well as easy, single-digit dispensing of the sanitizer by depressing a button on the watch face. The wearing of the present invention on one's body makes it difficult to misplace the contained sanitizer. This ensures one will always have a supply of hand sanitizer nearby. Additionally, the present invention can disperse hand sanitizer without having to be removed. This further prevents the present invention from being misplaced. Two-hand handling of a sanitizer bottle is no longer necessary.

### BRIEF DESCRIPTION OF THE DRAWINGS

FIG. 1 is a front-left perspective view of the present invention.

FIG. 2 is a right view of the present invention.

FIG. 3 is a front-right perspective view of a fluid receptacle of the present invention.

FIG. 4 is a top view of a fluid receptacle of the present invention.

FIG. 5 is an exploded view of a fluid receptacle of the present invention.

FIG. 6 is a top view of a holster of the present invention.

FIG. 7 is a right view of a holster of the present invention.

### DETAILED DESCRIPTION OF THE INVENTION

All illustrations of the drawings are for the purpose of describing selected versions of the present invention and are not intended to limit the scope of the present invention.

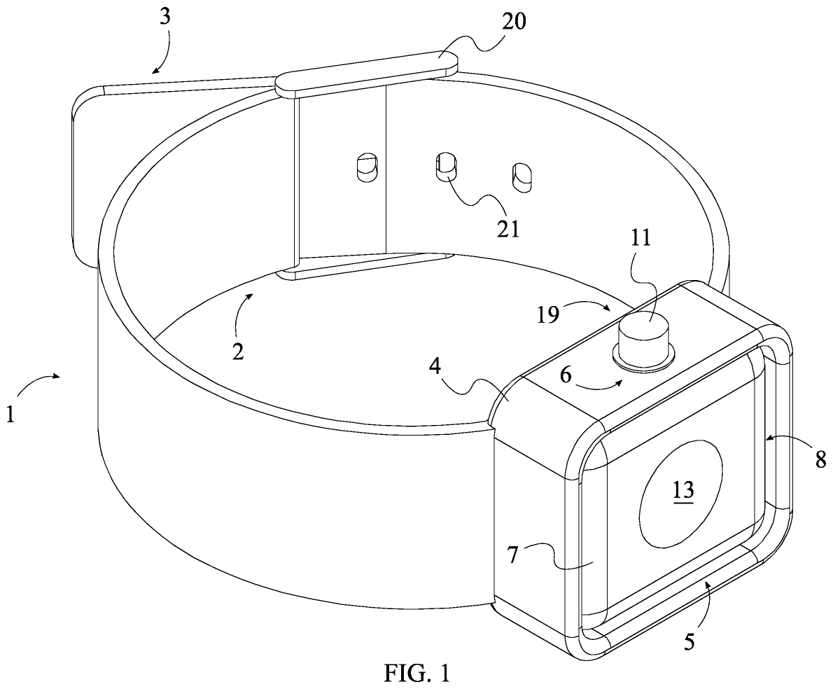

The present invention is a wearable sanitization device that is used to store and dispense sanitizing fluid upon a wearer's hands. The present invention is also configured to be worn upon a user's wrist or forearm. The present invention may comprise a wearable band 1, a holster 4, a fluid reservoir 7, a fluid dispenser 11, and a primary actuator 13, as shown in FIG. 1. The wearable band 1 is a generally flexible strap that enables the user to attach the present invention to the user's forearm or wrist. The holster 4 is a semirigid unit shaped to comfortably contain the fluid reservoir 7. The fluid reservoir 7 is a container that supports a volume of fluid, especially sanitizing fluid such as hand sanitizer. The fluid dispenser 11 denotes the mechanism for transporting fluid out of the fluid reservoir 7. The primary actuator 13 relates to a device that enables selective dispensation of fluid through the fluid dispenser 11.

The general configuration of the aforementioned components allows the present invention to efficiently and effectively retain sanitizing fluid for selective dispensation.

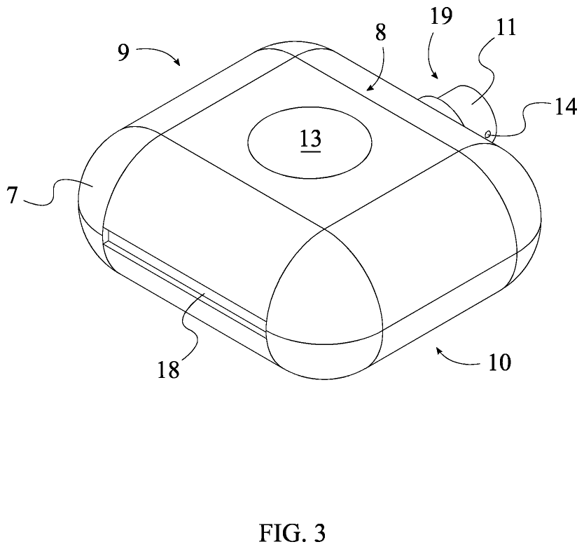

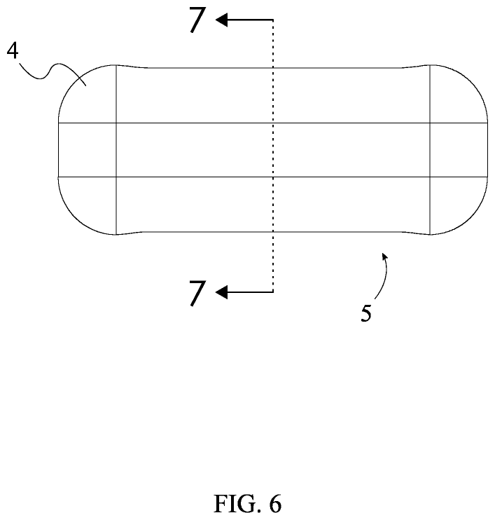

The wearable band 1 comprises a first band portion 2 and a second band portion 3. The first band portion 2 is an extended segment of the wearable band 1. Similarly, the second band portion 3 is also an extended segment of the wearable band 1. The holster 4 comprises a primary opening 5 and a secondary opening 6. The primary opening 5 is a cut into the holster 4 which enables addition and removal of the fluid reservoir 7, as shown in FIG. 6. The secondary opening 6 is a cut into the holster 4 that enables support of the fluid dispenser 11. The fluid reservoir 7 comprises an exposed face 8. The exposed face 8 relates to a surface of the fluid reservoir 7 which is physically accessible to a wearer during use. The fluid dispenser 11 comprises a valved outlet 12. The valved outlet 12 is an opening through which fluid may be selectively transmitted. The first band portion 2 may be connected adjacent to the holster 4. This arrangement enables the first band portion 2 to support the holster 4. The second first band portion 2 may be connected adjacent to the holster 4, opposite the first band portion 2. This arrangement enables the second band portion 3 to support the holster 4 in conjunction with the first band portion 2. The primary opening 5 and the secondary opening 6 may be positioned in between the first band portion 2 and the second band portion 3. In this way, the first band portion 2 and the second band portion 3 are positioned away from the holster 4, thus enabling appropriate arrangement of the fluid reservoir 7 and the fluid dispenser 11. The primary opening 5 and the secondary opening 6 may be positioned perpendicular to each other. Thus, the primary opening 5 and the secondary opening 6 are appropriately positioned for mounting of components. The fluid reservoir 7 may be mounted within the holster 4. The fluid holster 4 is preferably mounted through the primary opening 5, thus enabling intuitive access to the fluid reservoir 7. The exposed face 8 may be positioned adjacent to the primary opening 5. This arrangement results in the exposed face 8 being physically accessible to the user during the preferred usage of the present invention. The fluid dispenser 11 may traverse through the secondary opening 6 into the fluid reservoir 7. In this way, fluid contained in the fluid reservoir 7 and supported by the holster 4 may be dispensed through the second opening. The fluid dispenser 11 may be in fluid communication with the fluid reservoir 7. This arrangement enables fluid to flow from the fluid reservoir 7 out through the fluid dispenser 11. The valved outlet 12 may be externally positioned to the holster 4. Thus, the valved outlet 12 controls the flow of fluid out of the holster 4. The primary actuator 13 may be integrated into the exposed face 8, as shown in FIG. 3. In this way, the user may access the primary actuator 13 by interacting with the exposed face 8. The primary actuator 13 may be operatively coupled to the valved outlet 12, wherein the primary actuator 13 is used to selectively open the valved outlet 12. Thus, the user may control the flow of fluid from the fluid reservoir 7 out onto the user's palms and fingers.

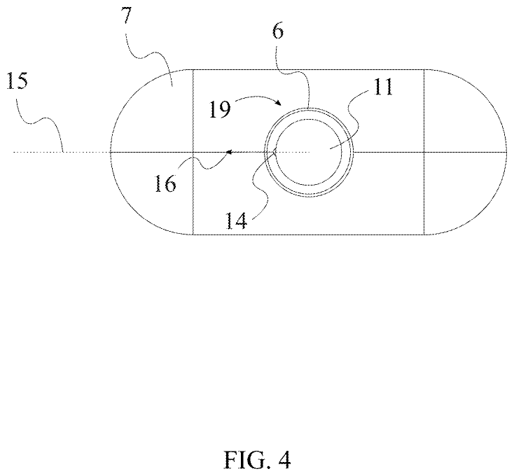

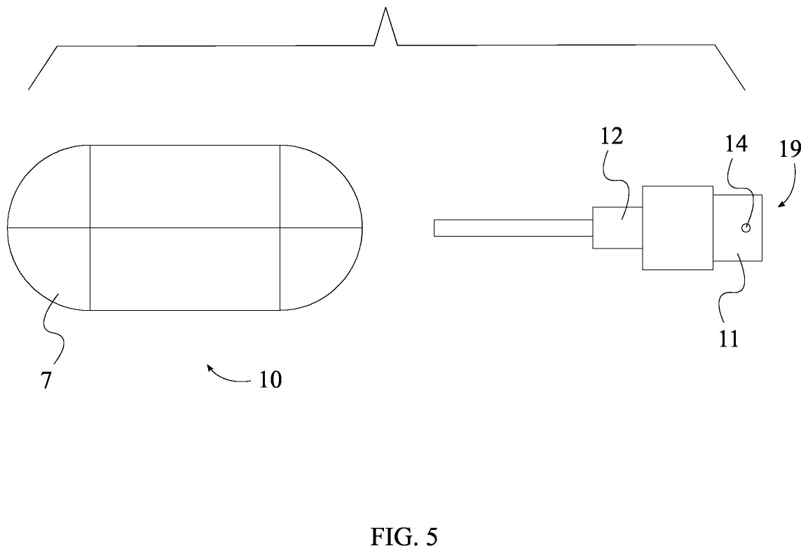

Many hand sanitizers are best applied through small particulates. To this end, the present invention may further comprise an atomizer 14, as shown in FIG. 5. The atomizer 14 is a small opening which disperses liquid particles into an aerated spray through application of pressure. The fluid reservoir 7 may comprise a first side 9 and a second side 10. The first side 9 is a wall or surface that denotes an extreme end of the fluid reservoir 7. Similarly, the second side 10 is a wall or surface that denotes an extreme end of the fluid reservoir 7. The first side 9 may be positioned coincident with the fluid dispenser 11. This arrangement allows the first side 9 to contact the fluid dispenser 11. The second side 10 may be positioned opposite to the first side 9 about the fluid reservoir 7. This arrangement enables establishment of an axis perpendicular to both the first side 9 and the second side 10. A spray axis 15 may traverse through the first side 9 and the second side 10, parallel to the exposed face 8. In this way, the spray axis 15 is appropriately positioned with respect to the fluid reservoir 7. The atomizer 14 may be mounted onto the fluid dispenser 11, adjacent to the valved outlet 12. Thus, the atomizer 14 is positioned to affect fluid exiting the fluid dispenser 11. The valved outlet 12 may be in fluid communication with the atomizer 14. In this way, fluid traversing through the valved outlet 12 is atomized before contacting the skin of the user. A spray direction 16 of the atomizer 14 may be oriented parallel with the spray axis 15, as shown in FIG. 4. This arrangement ensures that the spray direction 16 is advantageously positioned for dispensation of sanitizing fluid.

The user may benefit from the employment of an intuitive actuator. To this end, the primary actuator 13 may be a press button. Such an actuator ensures that a user can easily and conveniently dispense fluid through the fluid dispenser 11.

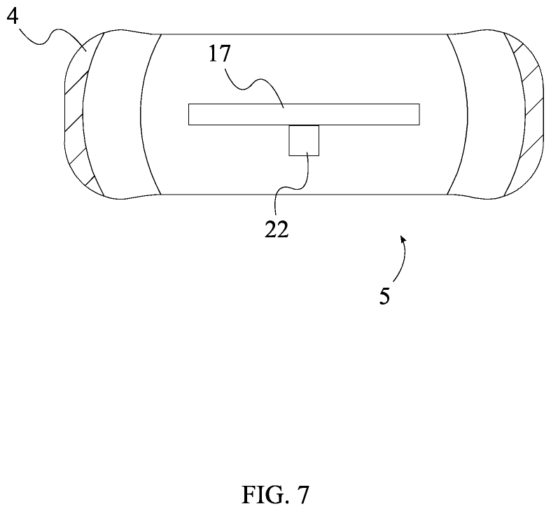

The fluid reservoir 7 is optimally connected securely within the holster 4 during use. To this end, the present invention may further comprise an engagement protrusion 17, as shown in FIG. 7. The engagement protrusion 17 relates to a semirigid segment that provides a ridge for interaction with the engagement cavity 18. The engagement cavity 18 relates to a cut or channel that provides a space into which the engagement protrusion 17 may fit. The engagement protrusion 17 may be connected within the holster 4. This arrangement allows the engagement protrusion 17 to join the holster 4 to the engagement cavity 18. The engagement cavity 18 may be integrated into the fluid reservoir 7. Thus, the engagement cavity 18 enables the fluid reservoir 7 to join to the engagement protrusion 17. The engagement protrusion 17 may be releasably attached into the engagement cavity 18. In this way, the fluid reservoir 7 is more securely connected to the holster 4 during use.

A user of the present invention may wish for an alternative mechanism for dispensing sanitizing fluid contained within the fluid reservoir 7. To provide for this, the present invention may further comprise a secondary actuator 19, as shown in FIG. 5. The secondary actuator 19 relates to a mechanism that enables selective release of fluid through the fluid dispenser 11. The secondary actuator 19 may be integrated into the fluid dispenser 11. This arrangement allows the secondary actuator 19 to affect the transfer of fluid through the fluid dispenser 11. The secondary actuator 19 is operatively coupled to the valved outlet 12, wherein the secondary actuator 19 is used to selectively open the valved outlet 12. Thus, the secondary actuator 19 may enable the user to dispense fluid onto the user's hand.

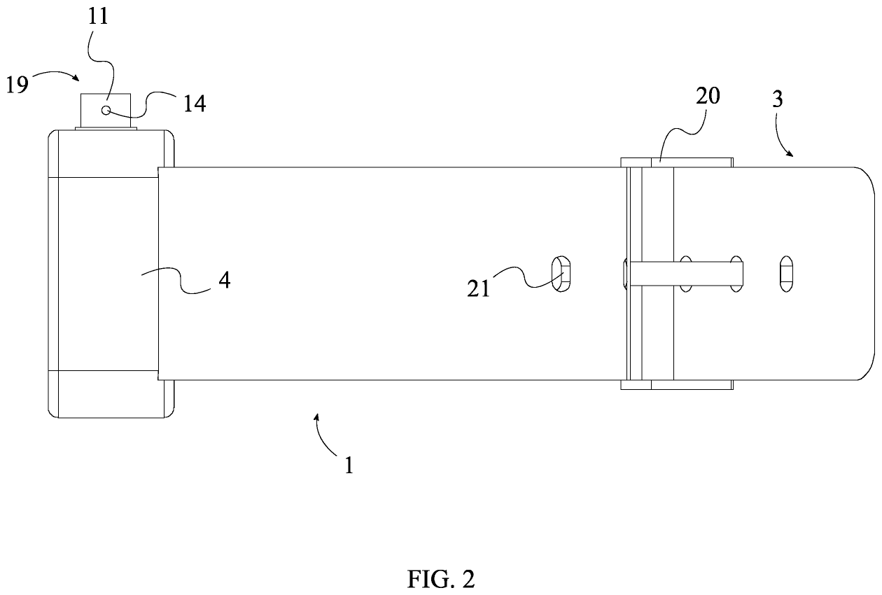

The user of the present invention may wish to secure the wearable band 1 onto the user's body, especially onto the user's wrist or forearm area. To this end, the present invention may further comprise a clasp 20 and a receiver 21, as shown in FIG. 2. The clasp 20 is a mechanism that enables retention of the wearable band 1. The receiver 21 is a segment that enables secure connection with the clasp 20. The clasp 20 may be terminally connected to the first band portion 2, opposite to the holster 4. This arrangement positions the clasp 20 advantageously for securing the receiver 21 opposite the holster 4. The receiver 21 may be terminally connected to the second band portion 3, opposite to the holster 4. This arrangement enables the user to manipulate the connection of the clasp 20 and the receiver 21. The receiver 21 may be engaged to the clasp 20. In this way, the user may adjust the size of the wearable band 1 through manipulation of the clasp 20 and the receiver 21.

The wearable band 1 is optimally made of material that enhances comfort without compromising durability and fatigue resistance. To this end, the wearable band 1 may be made of a flexible polymeric material. Such material may be rubber or a variety of polymeric blends capable of providing necessary support.

A user of the present invention may wish to dispense fluid on regular timed intervals. To this end, the present invention may further comprise a timer 22, as shown in FIG. 7. The timer 22 is a device which periodically activates at predetermined intervals. The timer 22 may be connected within the holster 4. This arrangement allows the timer 22 to be positioned appropriately to affect the fluid dispenser 11. The timer 22 may be operatively integrated into the fluid dispenser 11, wherein the timer 22 is used to periodically prompt the fluid dispenser 11 to open the valved outlet 12. Thus, the timer 22 may be used to dispense sanitizing fluid onto a user's hands at desirable intervals.

Although the invention has been explained in relation to its preferred embodiment, it is to be understood that many other possible modifications and variations can be made without departing from the spirit and scope of the invention as hereinafter claimed.

What is claimed is:

1. A wearable sanitization device comprises:
a wearable band;
a holster;
a fluid reservoir;
a fluid dispenser;
a primary actuator;
the wearable band comprises a first band portion and a second band portion;
the holster comprises a primary opening and a secondary opening;
the fluid reservoir comprises an exposed face;
the fluid dispenser comprises a valved outlet;
the first band portion being connected adjacent to the holster;
the second band portion being connected adjacent to the holster, opposite the first band portion;
the primary opening and the secondary opening being positioned in between the first band portion and the second band portion;
the primary opening and the secondary opening being positioned perpendicular to each other;
the fluid reservoir being mounted within the holster;
the exposed face being positioned adjacent to the primary opening;
the fluid dispenser traversing through the secondary opening into the fluid reservoir;
the fluid dispenser being in fluid communication with the fluid reservoir;
the valved outlet being externally positioned to the holster;
the primary actuator being integrated into the exposed face; and
the primary actuator being operatively coupled to the valved outlet, wherein the primary actuator is used to selectively open the valved outlet.

2. The wearable sanitization device as claimed in claim 1 comprises:
an atomizer;
the fluid reservoir comprises a first side and a second side;
the first side being positioned coincident with the fluid dispenser;
the second side being positioned opposite to the first side about the fluid reservoir;
a spray axis traversing through the first side and the second side, parallel to the exposed face;
the atomizer being mounted onto the fluid dispenser, adjacent to the valved outlet;
the valved outlet being in fluid communication with the atomizer; and
a spray direction of the atomizer being oriented parallel with the spray axis.

3. The wearable sanitization device as claimed in claim 1, wherein the primary actuator is a press button.

4. The wearable sanitization device as claimed in claim 1 comprises:
an engagement protrusion;
an engagement cavity;
the engagement protrusion being connected within the holster;
the engagement cavity being integrated into the fluid reservoir; and
the engagement protrusion being releasably attached into the engagement cavity.

5. The wearable sanitization device as claimed in claim 1 comprises:
a secondary actuator;
the secondary actuator being integrated into the fluid dispenser; and
the secondary actuator being operatively coupled to the valved outlet, wherein the secondary actuator is used to selectively open the valved outlet.

6. The wearable sanitization device as claimed in claim 1 comprises:
a clasp;
a receiver;
the clasp being terminally connected to the first band portion, opposite to the holster;
the receiver being terminally connected to the second band portion, opposite to the holster; and
the receiver being engaged to the clasp.

7. The wearable sanitization device as claimed in claim 1, wherein the wearable band is made of a flexible polymeric material.

8. The wearable sanitization device as claimed in claim 1 comprises:
a timer;
the timer being connected within the holster; and
the timer being operatively integrated into the fluid dispenser, wherein the timer is used to periodically prompt the fluid dispenser to open the valved outlet.

9. A wearable sanitization device comprises:
a wearable band;
a holster;
a fluid reservoir;
a fluid dispenser;
a primary actuator;
an engagement protrusion;
an engagement cavity;
the wearable band comprises a first band portion and a second band portion;
the holster comprises a primary opening and a secondary opening;
the fluid reservoir comprises an exposed face;
the fluid dispenser comprises a valved outlet;
the first band portion being connected adjacent to the holster;
the second band portion being connected adjacent to the holster, opposite the first band portion;
the primary opening and the secondary opening being positioned in between the first band portion and the second band portion;
the primary opening and the secondary opening being positioned perpendicular to each other;
the fluid reservoir being mounted within the holster;
the exposed face being positioned adjacent to the primary opening;
the fluid dispenser traversing through the secondary opening into the fluid reservoir;
the fluid dispenser being in fluid communication with the fluid reservoir;
the valved outlet being externally positioned to the holster;
the primary actuator being integrated into the exposed face;
the primary actuator being operatively coupled to the valved outlet, wherein the primary actuator is used to selectively open the valved outlet;
the engagement protrusion being connected within the holster;
the engagement cavity being integrated into the fluid reservoir; and
the engagement protrusion being releasably attached into the engagement cavity.

10. The wearable sanitization device as claimed in claim 9 comprises:
an atomizer;
the fluid reservoir comprises a first side and a second side;
the first side being positioned coincident with the fluid dispenser;
the second side being positioned opposite to the first side about the fluid reservoir;
a spray axis traversing through the first side and the second side, parallel to the exposed face;
the atomizer being mounted onto the fluid dispenser, adjacent to the valved outlet;
the valved outlet being in fluid communication with the atomizer; and
a spray direction of the atomizer being oriented parallel with the spray axis.

11. The wearable sanitization device as claimed in claim 9, wherein the primary actuator is a press button.

12. The wearable sanitization device as claimed in claim 9 comprises:
a secondary actuator;
the secondary actuator being integrated into the fluid dispenser; and
the secondary actuator being operatively coupled to the valved outlet, wherein the secondary actuator is used to selectively open the valved outlet.

13. The wearable sanitization device as claimed in claim 9 comprises:
a clasp;
a receiver;
the clasp being terminally connected to the first band portion, opposite to the holster;
the receiver being terminally connected to the second band portion, opposite to the holster; and
the receiver being engaged to the clasp.

14. The wearable sanitization device as claimed in claim 9, wherein the wearable band is made of a flexible polymeric material.

15. The wearable sanitization device as claimed in claim 9 comprises:
a timer;
the timer being connected within the holster; and
the timer being operatively integrated into the fluid dispenser, wherein the timer is used to periodically prompt the fluid dispenser to open the valved outlet.

16. A wearable sanitization device comprises:
a wearable band;
a holster;
a fluid reservoir;
a fluid dispenser;
a primary actuator;
an engagement protrusion;
an engagement cavity;
an atomizer;
a secondary actuator;
the wearable band comprises a first band portion and a second band portion;
the holster comprises a primary opening and a secondary opening;
the fluid reservoir comprises an exposed face;
the fluid dispenser comprises a valved outlet;
the fluid reservoir comprises a first side and a second side;
the first band portion being connected adjacent to the holster;
the second band portion being connected adjacent to the holster, opposite the first band portion;
the primary opening and the secondary opening being positioned in between the first band portion and the second band portion;
the primary opening and the secondary opening being positioned perpendicular to each other;
the fluid reservoir being mounted within the holster;
the exposed face being positioned adjacent to the primary opening;
the fluid dispenser traversing through the secondary opening into the fluid reservoir;
the fluid dispenser being in fluid communication with the fluid reservoir;
the valved outlet being externally positioned to the holster;
the primary actuator being integrated into the exposed face;
the primary actuator being operatively coupled to the valved outlet, wherein the primary actuator is used to selectively open the valved outlet;
the engagement protrusion being connected within the holster;
the engagement cavity being integrated into the fluid reservoir;
the engagement protrusion being releasably attached into the engagement cavity;
the first side being positioned coincident with the fluid dispenser;
the second side being positioned opposite to the first side about the fluid reservoir;
a spray axis traversing through the first side and the second side, parallel to the exposed face;
the atomizer being mounted onto the fluid dispenser, adjacent to the valved outlet;
the valved outlet being in fluid communication with the atomizer;
a spray direction of the atomizer being oriented parallel with the spray axis;
the secondary actuator being integrated into the fluid dispenser; and
the secondary actuator being operatively coupled to the valved outlet, wherein the secondary actuator is used to selectively open the valved outlet.

17. The wearable sanitization device as claimed in claim 16, wherein the primary actuator is a press button.

18. The wearable sanitization device as claimed in claim 16 comprises:
a clasp;
a receiver;
the clasp being terminally connected to the first band portion, opposite to the holster;
the receiver being terminally connected to the second band portion, opposite to the holster; and
the receiver being engaged to the clasp.

19. The wearable sanitization device as claimed in claim 16, wherein the wearable band is made of a flexible polymeric material.

20. The wearable sanitization device as claimed in claim 16 comprises:
a timer;
the timer being connected within the holster; and
the timer being operatively integrated into the fluid dispenser, wherein the timer is used to periodically prompt the fluid dispenser to open the valved outlet.
